(19) United States
(12) Patent Application Publication
(10) Pub. No.: US 2024/0041450 A1
(43) Pub. Date: Feb. 8, 2024

(54) TEN-DIMENSIONAL BARBED SURGICAL THREAD

(71) Applicant: Eurothreads LLC, Hudson, WI (US)

(72) Inventors: Tamara Moore, Hudson, WI (US); Laurie Wall, Hudson, WI (US)

(21) Appl. No.: 18/489,632

(22) Filed: Oct. 18, 2023

Related U.S. Application Data
(63) Continuation-in-part of application No. 17/240,964, filed on Apr. 26, 2021, now abandoned.
(60) Provisional application No. 63/015,608, filed on Apr. 26, 2020.

(51) Int. Cl. A61B 17/06

(52) U.S. Cl. CPC A61B 17/06166; A61B 2017/06176; A61B 2017/00792

(57) ABSTRACT

A method of using a ten-dimensional barbed surgical thread to perform one of a chest lift, buttocks lift and heavy facial tissue lift. A ten-dimensional barbed surgical thread is provided that includes a central core, ten first barbs and ten second barbs. The central core has a circumference, a central core proximal end and a central core distal end that is opposite the central core proximal end. Each of the first barbs occupies about 36 degrees of the circumference of the central core. The ten first barbs are positioned radially adjacent to each other around the circumference of the central core and obliquely extend from the central core. Each of the ten first barbs has a first barb distal end and a first barb proximal end. Each of first barbs is oriented so that the first barb distal end is closer to the central core distal end than the first barb proximal end. Each of the second barbs occupies about 36 degrees of the circumference of the central core. The ten second barbs are positioned radially adjacent to each other around the circumference of the central core and obliquely extend from the central core. The ten second barbs are closer to the central core distal end than the ten first barbs. Each of the ten second barbs has a second barb distal end and a second barb proximal end. Each of the second barbs is oriented so that the second barb distal end is closer to the central core proximal end than the second barb proximal end. The ten-dimensional barbed surgical thread is inserted into a dermis on a patient. The ten first barbs are engaged in the dermis to lift the dermis to a lifted position. The ten second barbs are engaged in the dermis to anchor the dermis in the lifted position and complete the one of the chest lift, buttocks lift and heavy facial tissue lift.

### REFERENCE TO RELATED APPLICATION

This application is a continuation-in-part of U.S. application Ser. No. 17/240,964, which was filed on Apr. 26, 2021, and claims priority to U.S. Applic. No. 63/015,608, which was filed on Apr. 26, 2020. The contents of which are incorporated herein by reference.

### FIELD OF THE INVENTION

The invention relates generally to thread for surgical applications. More particularly, the invention relates to ten-dimensional barbed surgical thread.

### BACKGROUND OF THE INVENTION

A variety of aesthetic medical techniques have been developed to enhance the appearance of a person's skin and, in particular, the appearance of the person's face. These techniques range from cutting and tightening of the skin to injecting compositions into the skin such as to reduce wrinkles. Despite these advances, there is a continuing need to non-surgical techniques to improve a person's appearance.

Ruff, U.S. Pat. No. 5,342,376 describes a barbed tissue connector that is used in conjunction with approximating tissue in a wound to seal the wound. There is no teaching or suggestion in this patent to use the barbed tissue connector in conjunction with cosmetic or aesthetic surgery to cause lifting of tissue such as in conjunction with a chest lift, a buttocks lift or a heavy facial tissue lift. While this publication illustrates the barbs being provided in a variety of configuration, it does not teach or suggest each of the barbs occupying about 36 degrees of the circumference of the central core and positioning the barbs radially adjacent to each other around the circumference of the central core.

Ruff, U.S. Patent Publication No. 2005/0267531 describes a barbed suture and indicates that it may be used in conjunction with cosmetic procedures such as in Paragraph [0068]. While this publication illustrates the barbs being provided in a variety of configuration, it does not teach or suggest each of the barbs occupying about 36 degrees of the circumference of the central core and positioning the barbs radially adjacent to each other around the circumference of the central core. Because of the differences between the barb configuration used on this barbed suture and the ten-dimensional barbed surgical thread of this application, this publication describes implanting the barbed suture in a sinusoidal pattern to provide shock absorbing properties.

Wu, U.S. Patent Publication No. 2005/0240224 describes a barbed surgical thread that may be used in conjunction with cosmetic surgery. While this publication illustrates the barbs being provided in a variety of configuration, it does not teach or suggest each of the barbs occupying about 36 degrees of the circumference of the central core and positioning the barbs radially adjacent to each other around the circumference of the central core. Because of the differences between the barbed configuration described in this publication and the ten-dimensional barbed surgical thread of this application, this publication describes implanting the barbed surgical thread in a U-shaped pattern.

### SUMMARY OF THE INVENTION

An embodiment of the invention is directed to a ten-dimensional barbed surgical thread that includes a central core and ten first barbs. The central core has a circumference, a proximal end and a distal end that is opposite the proximal end. The ten first barbs are positioned around the circumference of the central core so that the first barbs are radially adjacent to each other and obliquely extend from the central core.

Another embodiment of the invention is directed to a ten-dimensional barbed surgical thread a central core, ten first barbs and ten second barbs. The central core has a circumference, a proximal end and a distal end that is opposite the proximal end. The ten first barbs are positioned around the circumference of the central core and obliquely extend from the central core. Each of the ten first barbs has a distal end and a proximal end. The distal end of one of the first barbs is closer to the proximal end of the central core than the proximal end of the one of the second barbs. The ten second barbs are positioned around the circumference of the central core and obliquely extend from the central core. The ten second barbs are closer to the proximal end than the ten first barbs. Each of the ten second barbs has a distal end and a proximal end. The distal end of one of the second barbs is closer to the distal end of the central core than the proximal end of the one of the second barbs.

Another embodiment of the invention is directed to a method of using a ten-dimensional barbed surgical thread. A ten-dimensional barbed surgical thread is provided that includes a central core, ten first barbs and ten second barbs. The central core has a circumference, a proximal end and a distal end that is opposite the proximal end. The ten first barbs are positioned around the circumference of the central core and obliquely extend from the central core. Each of the ten first barbs has a distal end and a proximal end. The distal end of one of the first barbs is closer to the proximal end of the central core than the proximal end of the one of the second barbs. The ten second barbs are positioned around the circumference of the central core and obliquely extend from the central core. The ten second barbs are closer to the proximal end than the ten first barbs. Each of the ten second barbs has a distal end and a proximal end. The distal end of one of the second barbs is closer to the distal end of the central core than the proximal end of the one of the second barbs. The ten-dimensional barbed surgical thread is inserted into a dermis on a patient. The ten first barbs engage the dermis to lift the dermis to a lifted position. The ten second barbs engage the dermis to anchor the dermis in the lifted position.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other Like reference numerals designate corresponding similar parts.

FIG. 1 is a side view of a ten-dimensional barbed surgical thread according to an embodiment of the invention.

FIG. 2 is a cross-sectional overview of the ten-dimensional barbed surgical thread.

FIG. 3 is a photograph of a female's face where a medical procedure has been performed on a right side of the female's face.

FIG. 4 is a photograph of another female's face where a medical procedure has been performed on a right side of the female's face.

FIG. 5 is a photograph of another female's face where a medical procedure has been performed on a right side of the female's face.

FIG. 6 is a photograph of a male's torso prior to a medical procedure.

FIG. 7 is a photograph of the male's torso from FIG. 6 after the medical procedure has been performed.

FIG. 8 is a photograph of another male's torso prior to a medical procedure.

FIG. 9 is a photograph of the male's torso from FIG. 8 after the medical procedure has been performed.

### DETAILED DESCRIPTION OF THE INVENTION

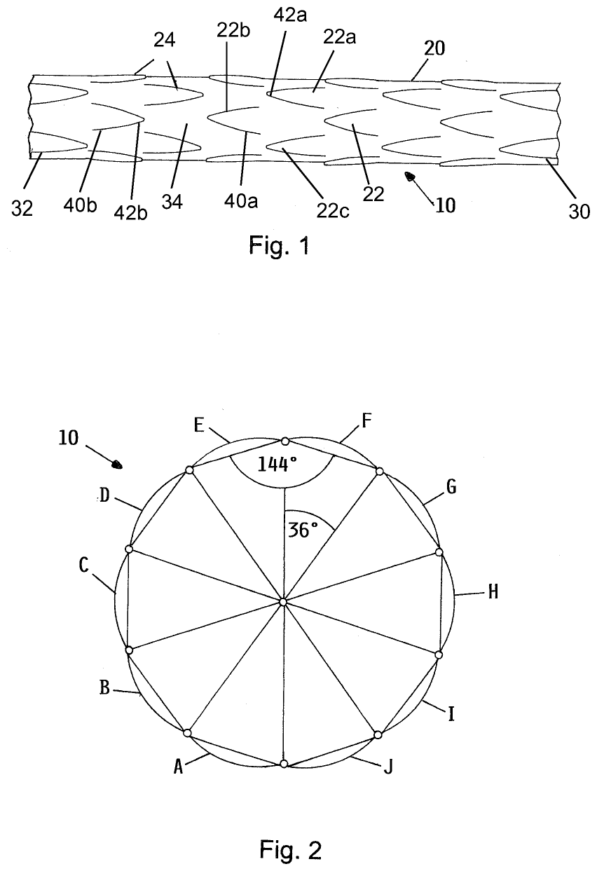

The invention is directed to a ten-dimensional barbed surgical thread as illustrated at 10 in FIG. 1. The ten-dimensional barbed surgical thread 10 is particularly suited for use in cosmetic enhancement procedures. The ten-dimensional barbed surgical thread 10 includes a central core 20 from which a plurality of first barbs 22 extend.

An advantage of the ten-dimensional barbed surgical thread 10 over the prior art barbed surgical threads is that the ten-dimensional barbed surgical thread 10 of this invention produces multiple effects once introduced into the dermis.

The ten-dimensional barbed surgical thread 10 is particularly suited for use in cosmetic and aesthetic procedures in which it is desired to lift particularly heavy tissue such as on the chest, buttocks and heavy facial tissue in a minimally invasive manner.

Such procedures are quite different that convention surgical procedures because with the minimally invasive procedures, it is desired for the patient on which the ten-dimensional barbed surgical thread 10 is used can leave the clinic or other location where the procedure is performed shortly after the procedure is performed.

Additionally, the minimally invasive procedures done using the ten-dimensional barbed surgical thread 10 enable the patient to return to typical daily activities relatively soon after the procedure has been performed. The recovery from surgical procedures using the ten-dimensional barbed surgical thread 10 is thereby considerably shorter that the recovery from conventional cosmetic procedures.

As compared to the six-dimensional barbed surgical thread and the eight-dimensional barbed surgical thread described in the recently filed patent applications owned by the assignee of this patent application, the ten-dimensional barbed surgical thread provides superior performance when used in conjunction with heavier and/or thicker skin. In particular, the ten-dimensional barbed surgical thread provides superior performance when used in conjunction with chest, buttocks and heavy facial tissue. These results are possible because the ten-dimensional barbed surgical thread 10 has more points of contact than the six-dimensional barbed surgical thread and eight-dimensional barbed surgical thread.

These effects fall into three major categories: (1) instant skin lifting through mechanical effects, (2) cellular renewal through collagen stimulation and neovascularization to improve skin texture, fine lines and elasticity, and (3) skin tightening by contracting fat tissue.

The ten-dimensional barbed surgical thread 10 is a medical device that in certain embodiments consists of a monofilament surgical suture. The ten-dimensional barbed surgical thread 10 may be fabricated from a variety of materials. In certain embodiments, the materials used to fabricate the ten-dimensional barbed surgical thread is absorbable inside of a human body. A non-limiting example of a suitable material for fabricating the ten-dimensional barbed surgical thread 10 is a polydioxanone (PDO)/polycaprolactone (PCL) polymer.

In certain embodiments, a cut is used to form each of the first barbs 22. A variety of cutting techniques may be used. In certain embodiments, the ten-dimensional barbed surgical thread 10 is fabricated by laser cutting into the surgical suture octagonally at an angle of about 36 degrees as illustrated in FIG. 2. The locations of the six cuts are indicated by letters A-J.

Using such a configuration, the ten first barbs 22 are radially adjacent to each other when going once around a circumference of the ten-dimensional barbed surgical thread 10. While in certain embodiments, the first barbs 22 are immediately radially adjacent to each other such as illustrated in FIG. 2, it is possible that there can be a spacing between adjacent first barbs 22. As used herein, the spacing between adjacent first barbs 22 is less than about 20 percent of the width of the widest portion of the first barbs 22. In other embodiments, the spacing between adjacent first barbs 22 is less than about 10 percent of the width of the widest portion of the first barbs 22.

Using the preceding configuration enhances the potential of contacts between the first barbs 22 after insertion into the dermis regardless of the orientation of the ten-dimensional barbed surgical thread 10, which reduces the potential of the dermis sagging after insertion of the ten-dimensional barbed surgical thread 10.

The cutting that is done to form the first barb 22 is done at an angle to the surface of the central core 20. In certain configurations, the first barbs 22 are oblique, notched protrusions formed into a surgical suture at an angle of between about 10 degrees and about 15 degrees. In other embodiments, the angle of the cut to form the first barb 22 is about 12.5 degrees. Cutting in this manner causes a width of the first barb 22 decreases when moving from the proximal end 40a to the distal end 42a. In certain embodiments, the distal end 42a of the first barb 22 is pointed.

In certain embodiments, a ratio of a length of the first barb to a width of the first barb is between about 2:1 and about 5:1. Forming the first barbs 22 with the length to width ratio in this range provides the first barbs 22 with sufficient strength for the first barbs 22 to perform the desired functions during the insertion process and after insertion.

In certain embodiments, the adjacent first barbs 22 are offset in a direction moving from a distal end 32 to a proximal end 30 of the central core 20 as illustrated in FIG. 1. In one such embodiment, a distal end 42a of one first barb 22a is approximately aligned with a proximal end 40a of an adjacent barb 22b around a circumference of the central core 20, as illustrated in FIG. 1. Additionally, the distal end 42a of every other first barb 22a, 22c is approximately aligned around a circumference of the central core 20, as illustrated in FIG. 1.

The first barbs 22 have a proximal end 40a and a distal end 42a. The proximal end 40a is where the first barb 22 attaches to the central core 20. The distal end 42a is opposite the proximal end 40.

The plurality of first barbs 22 are cut at intervals of about 36 degrees on ten sides of the ten-dimensional barbed surgical thread 10. Using such a configuration causes adjacent first barbs 22 to appear to be next to each other when viewed from the cross-sectional view, which is illustrated in FIG. 2, even though the adjacent first barbs 22 are offset from each other as illustrated in FIG. 1. Using this configuration for the first barbs 22 provides the ten-dimensional barbed surgical thread 10 with enhanced performance when used in conjunction with performing a chest lift, a buttocks lift or a heavy facial tissue lift as is described in more detail herein. In certain embodiments, the distance between adjacent first barbs 22 is about 1.5 millimeters, which is closer than the conventional spacing of about 1.8 millimeters that is used in the prior art surgical thread that is used when performing facelifts.

The plurality of first barbs 22 are oriented such that the distal end 42a of each first barb 22 is closer to the central core distal end 32 than the proximal end 40a of each first barb 22. As described in more detail below, a primary function of the plurality of first barbs 22 is lifting.

In certain embodiments, the plurality of first barbs 22 occupy greater than about ½ of a length of the ten-dimensional barbed surgical thread 10. In other embodiments, the plurality of first barbs 22 occupy about ⅔ of the length of the ten-dimensional barbed surgical thread 10.

The ten-dimensional barbed surgical thread 10 is typically inserted so that the plurality of first barbs 22 are facing upwardly. The configuration of the plurality of first barbs 22 on the ten-dimensional barbed surgical thread 10 thereby allows for most of the ten-dimensional barbed surgical thread 10 to assist in lifting of the tissue.

The ten-dimensional barbed surgical thread 10 also includes a plurality of second barbs 24 proximate the central core proximal end 30. The ten-dimensional barbed surgical thread 10 is typically inserted so that the plurality of second barbs 24 are facing downwards. As described in more detail below, a primary function of the plurality of second barbs 24 is anchoring.

Similar to the first barbs 22, the plurality of second barbs 24 are cut at intervals of about 36 degrees on ten sides of the ten-dimensional barbed surgical thread 10. In certain embodiments, the distance between adjacent second barbs 24 is about 1.5 millimeters, which is closer than the conventional spacing of about 1.8 millimeters that is used in the prior art surgical thread that is used when performing facelifts.

Other than the orientation in an opposite direct, the second barbs 24 may be shaped similarly to the first barbs 22 and the second barbs 24 may be positioned on the ten-dimensional barbed surgical thread 10 similarly to the first barbs 22. The plurality of second barbs 24 are oriented such that the distal end 42b of each second barb 24 is closer to the central core proximal end 30 than the distal end 40b of each second barb 24.

In certain embodiments, the plurality of second barbs 24 occupies less than about ½ of the length of the ten-dimensional barbed surgical thread 10. In other embodiments, the plurality of second barbs 24 occupies about ⅓ of the length of the ten-dimensional barbed surgical thread 10.

In certain embodiments, there may be an intermediate region 34 of the ten-dimensional barbed surgical thread 10 that is intermediate the upwardly facing first barbs 22 and the downwardly facing second barbs 24 from which no barbs extend therefrom. This intermediate region 34 on the ten-dimensional barbed surgical thread 10 may have a length that is smaller than the length of the ten-dimensional barbed surgical thread 10 over which the upwardly extending first barbs 22 extend. The length of the intermediate region of the ten-dimensional barbed surgical thread 10 may be smaller than the length of the ten-dimensional barbed surgical thread 10 over which the downwardly extending second barbs 24 extend.

Unlike the prior art two-dimensional, three-dimensional and four-dimensional barbed surgical thread that limited the ability to engage tissue, the ten-dimensional barbed surgical thread 10 of this invention significantly improves the viability and uniformity of tissue engagement thus the sustainability of the desired result.

Additionally, upon insertion of the ten-dimensional barbed surgical thread 10 into the dermis, the dermis sustains minor injuries. These minor injuries engage the body's natural healing process and stimulate the skin cells to produce collagen and blood vessels, which improves skin microcirculation.

It has been found that in areas of the body that do not have bone structure beneath, such as the fatty area of the cheek, having more points of tissue attachment provides a greater degree of lift, greater points of injury and a larger amount of fiberblasting, which thereby results in greater collagen production.

The ten-dimensional barbed surgical 10 is inserted into the subcutaneous layer of the dermis. In certain embodiments, a cannula is used to insert the ten-dimensional barbed surgical thread 10 into the dermis. In other embodiments, the cannula is a rounded or L-tipped surgical steel cannula.

The ten-dimensional barbed surgical thread 10 is not attached to the surgical cannula and is introduced into the soft tissue through the tip of the cannula. Once the cannula reaches the desired end site, the cannula is rotated about 180 degrees mechanically engaging the ten-dimensional barbed surgical thread 10 at the distal (lower) end.

The cannula is then removed leaving only the ten-dimensional barbed surgical thread 10 in the dermis. Approximating the tissue up the ten-dimensional barbed surgical thread 10 (lifting the tissue onto each first barb) allows for each of the first barbs 22 to become securely engaged in the dermis. Once the desired result is achieved, the ten-dimensional barbed surgical thread 10 is anchored at the proximal (upper) end. Any excess portion of the ten-dimensional barbed surgical thread 10 is cut off and discarded.

The injection of ten-dimensional barbed surgical threads 10 is indicated for soft tissue augmentation where the insertion of surgical sutures is appropriate. The ten-dimensional barbed surgical threads 10 are used to lift, contour and volumize the skin. The implantation of the ten-dimensional barbed surgical threads 10 is indicated for subcutaneous (intradermal and hypodermal) implantation.

In operation, once the area to be treated is defined, and an appropriate examination is completed, the patient is seated. The treatment area should be prepped by cleansing and removing the topical anesthetic. If topical anesthetic is to be used, it is applied liberally to the treatment areas.

The appropriate thread packages required for the treatment area are opened and removed from the package. The thread is attached in accordance with the manufacturer's instructions. Proper use of the product(s) should minimize the chances of dislodging or breaking while injecting the thread.

Correct injection technique is important to the success of the treatment in achieving the desired results. The needle or cannula should be inserted into the treatment site with the tip ending up at an appropriate depth within the skin. The ten-dimensional barbed surgical thread 10 should then be released using a slow, steady withdrawal of the needle/cannula. Overcorrection, which is more threads than suggested or required, is generally not needed and is to be avoided.

Once the first thread is appropriately inserted, another thread is inserted into the next adjacent location, and the process is repeated. Care should be taken to adequately assess the entire area to be treated with the correct number of threads to ensure even and symmetrical distribution of the product.

Once the injection is completed, the treated areas should be gently massaged, setting the threads per instruction. More vigorous massage may result in additional swelling, bruising or dislodgement of the thread.

Unlike the prior art two-dimensional, three-dimensional and four-dimensional barbed surgical threads that are limited in their ability to engage tissue due to barbs being cut respectively on only 2, 3 or 4 sides of the thread. This greatly limits the number of points of engagement into soft tissue. It is this engagement that is necessary to achieve a sustainable lift.

With ten-dimensional barbed surgical threads 10, the presence of additional first barbs 22 more than double the points of tissue engagement of the four-dimensional barbed surgical thread. Regardless of how the ten-dimensional barbed surgical thread 10 is inserted into subcutaneous tissue, the additional first barbs 22 allows the ten-dimensional barbed surgical thread 10 to grab onto additional dermis resulting in a better lift.

The ten-dimensional barbed surgical thread that is fabricated from PDO alone or in combination with PCL is slowly absorbed after implantation into the patient. In certain embodiments, the ten-dimensional barbed surgical thread is resorbed over a time period of between about 180 days and about 240 days. After this time period, the ten-dimensional barbed surgical thread 10 is substantially absorbed into the surrounding tissue. As used herein, substantially absorbed means that there are only minimal traces remaining of the ten-dimensional barbed surgical thread 10.

After implantation, the proximal ends of the ten-dimensional barbed surgical threads 10 are trimmed so that the entirety of the remaining ten-dimensional barbed surgical threads are recessed below the surface of the person's skin.

### Examples

In the following example, the performance of the ten-dimensional barbed surgical thread according to this invention is evaluated. The subject on which the procedure was performed is a Caucasian female.

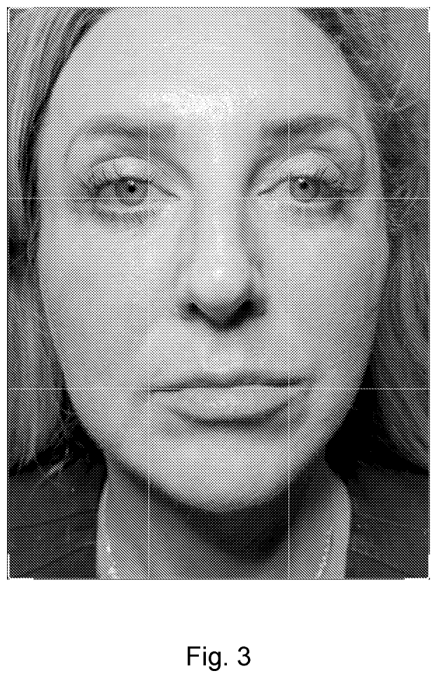

FIG. 3 illustrates the performance of the ten-dimensional barbed surgical thread. The left side of the female's face is prior to implantation of the ten-dimensional barbed surgical thread and the right side of the female's face is after implantation of the ten-dimensional barbed surgical thread. The ten-dimensional barbed surgical thread exhibits good results in lifting the right side of the female's face.

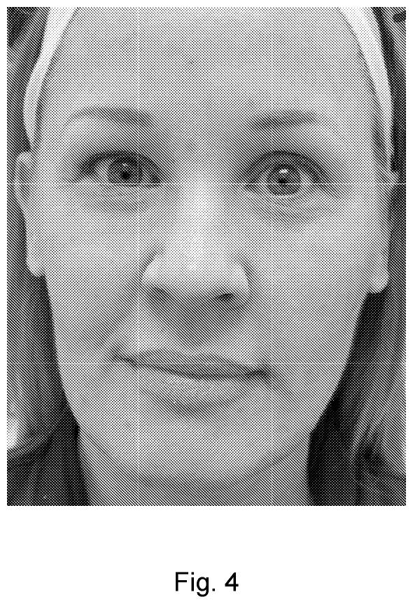

FIG. 4 illustrates the performance of the ten-dimensional barbed surgical thread. The left side of the female's face is prior to implantation of the ten-dimensional barbed surgical thread and the right side of the female's face is after implantation of the ten-dimensional barbed surgical thread. The ten-dimensional barbed surgical thread exhibits good results in lifting the right side of the female's face.

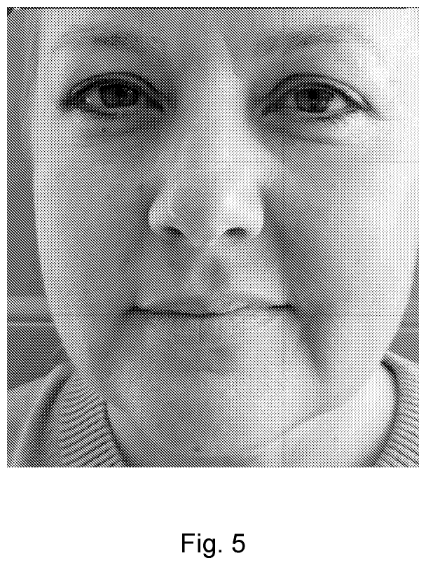

FIG. 5 illustrates the performance of the ten-dimensional barbed surgical thread. The left side of the female's face is prior to implantation of the ten-dimensional barbed surgical thread and the right side of the female's face is after implantation of the ten-dimensional barbed surgical thread. The ten-dimensional barbed surgical thread exhibits good results in lifting the right side of the female's face.

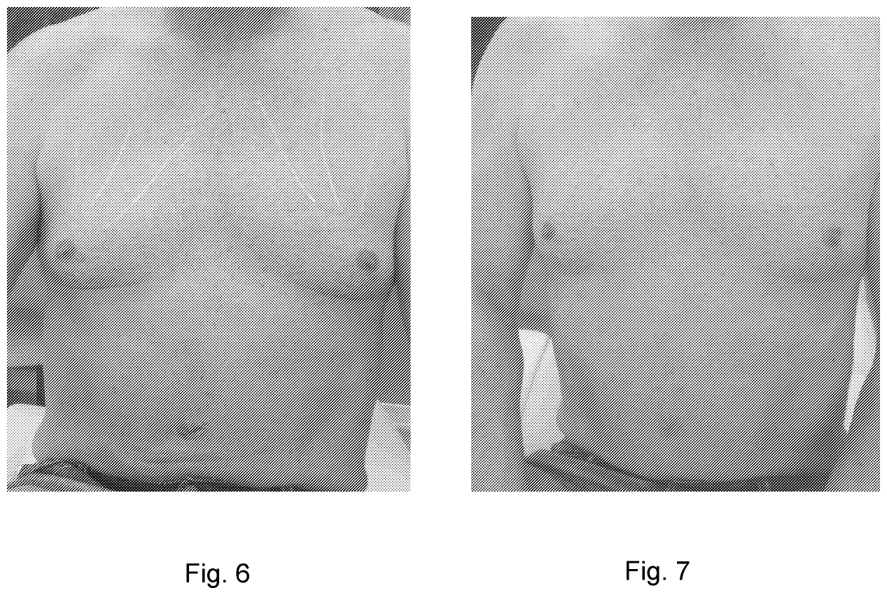

FIGS. 6 and 7 are before and after front photographs, respectively, of a male subject on which mastopexy was performed. The ten-dimensional barbed surgical thread exhibits good results in lifting the right side of the male's chest.

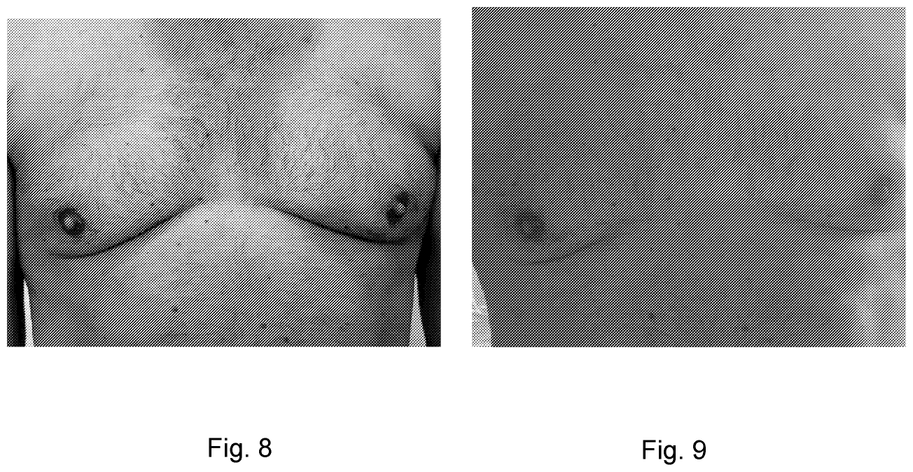

FIGS. 8 and 9 are before and after front photographs, respectively, of a male subject on which mastopexy was performed. The ten-dimensional barbed surgical thread exhibits good results in lifting the right side of the male's chest.

In the preceding detailed description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The preceding detailed description, therefore, is not to be taken in a limiting sense, and the scope of the present invention is defined by the appended claims.

It is contemplated that features disclosed in this application, as well as those described in the above applications incorporated by reference, can be mixed and matched to suit particular circumstances. Various other modifications and changes will be apparent to those of ordinary skill.

1. A method of using a ten-dimensional barbed surgical thread to perform one of a chest lift, buttocks lift and heavy facial tissue lift, wherein the method comprises:
providing a ten-dimensional barbed surgical thread comprising a central core, ten first barbs and ten second barbs, wherein the central core has a circumference, a central core proximal end and a central core distal end that is opposite the central core proximal end, wherein each of the first barbs occupies about 36 degrees of the circumference of the central core, wherein the ten first barbs are positioned radially adjacent to each other around the circumference of the central core and obliquely extend from the central core, wherein each of the ten first barbs has a first barb distal end and a first barb proximal end, wherein each of first barbs is oriented so that the first barb distal end is closer to the central core distal end than the first barb proximal end, wherein each of the second barbs occupies about 36 degrees of the circumference of the central core, wherein the ten second barbs are positioned radially adjacent to each other around the circumference of the central core and obliquely extend from the central core, wherein the ten second barbs are closer to the central core distal end than the ten first barbs, wherein each of the ten second barbs has a second barb distal end and a second barb proximal end and wherein each of the second barbs is oriented so that the second barb distal end is closer to the central core proximal end than the second barb proximal end; and
inserting the ten-dimensional barbed surgical thread into a dermis on a patient;
engaging the ten first barbs in the dermis to lift the dermis to a lifted position; and
engaging the ten second barbs in the dermis to anchor the dermis in the lifted position and complete the one of the chest lift, buttocks lift and heavy facial tissue lift.

2. The method of claim 1, wherein the ten first barbs occupy about ⅔ of a length of the ten-dimensional barbed surgical thread and wherein the ten second barbs occupy about ⅓ of the length of the ten-dimensional barbed surgical thread.

3. The method of claim 1, wherein adjacent barbs in the ten first barbs are offset in a direction extending between the proximal end and the distal end of the central core so that the first barb distal end of one of the ten first barbs is approximately aligned with the first barb proximal end of an adjacent one of the ten first barbs around the circumference of the central core and wherein every other of the ten first barbs is approximately aligned around the circumference of the central core.

4. The method of claim 1, wherein each of the first barbs occupies about 36 degrees of a circumference of the central core and wherein a cut is formed at an angle of between about 10 degrees and about 15 degrees with respect to a surface of the central core to form each of the first barbs.

5. The method of claim 1, wherein the ten-dimensional barbed surgical thread comprises a plurality of rows of the ten first barbs and wherein a spacing between the rows of the ten first barbs is less than about a length of one of the ten first barbs in a direction that extends between the central core proximal end and the central distal end.

6. The method of claim 1, wherein insertion of the ten-dimensional barbed surgical thread causes cellular renewal through collagen stimulation and neovascularization to improve skin texture.

7. The method of claim 1, wherein insertion of the ten-dimensional barbed surgical thread into the dermis causes skin tightening caused by contracting fat tissue.

8. The method of claim 1, wherein the ten-dimensional barbed surgical thread is inserted into the dermis using a cannula.

9. The method of claim 8, wherein the cannula is rotated to cause the ten second barbs to engage the dermis.

10. The method of claim 1, and after anchoring the dermis in the lifted position, cutting off a portion of the proximal end of the ten-dimensional barbed surgical thread that extends through a skin surface on the person patient.

11. A method of performing one of a chest lift, buttocks lift and heavy facial tissue lift, wherein the method comprises:
providing a ten-dimensional barbed surgical thread comprising a central core, ten first barbs and ten second barbs, wherein the central core has a circumference, a central core proximal end and a central core distal end that is opposite the central core proximal end, wherein each of the first barbs occupies about 36 degrees of the circumference of the central core, wherein the ten first barbs are positioned radially adjacent to each other around the circumference of the central core and obliquely extend from the central core, wherein each of the ten first barbs has a first barb distal end and a first barb proximal end, wherein each of first barbs is oriented so that the first barb distal end is closer to the central core distal end than the first barb proximal end, wherein each of the second barbs occupies about 36 degrees of the circumference of the central core, wherein the ten second barbs are positioned radially adjacent to each other around the circumference of the central core and obliquely extend from the central core, wherein the ten second barbs are closer to the central core distal end than the ten first barbs, wherein each of the ten second barbs has a second barb distal end and a second barb proximal end and wherein each of the second barbs is oriented so that the second barb distal end is closer to the central core proximal end than the second barb proximal end;
providing a cannula;
positioning the ten-dimensional surgical thread at least partially in the cannula; inserting the cannula into the dermis;
remove the cannula from the dermis while leaving the ten-dimensional surgical thread at least partially in the dermis;
engaging the ten first barbs in the dermis to lift the dermis to a lifted position; and
engaging the ten second barbs in the dermis to anchor the dermis in the lifted position and complete the one of the chest lift, buttocks lift and heavy facial tissue lift.

12. The method of claim 11, wherein the cannula is rotated to cause the ten second barbs to engage the dermis.

13. The method of claim 11, wherein the cannula is a rounded or L-tipped surgical steel cannula.

14. The method of claim 11, and after anchoring the dermis in the lifted position, cutting off a portion of the proximal end of the ten-dimensional barbed surgical thread that extends through a surface of the skin.
